(19) United States
(12) Patent Application Publication
(10) Pub. No.: US 2024/0037413 A1
(43) Pub. Date: Feb. 1, 2024

(54) COMPUTER-IMPLEMENTED METHOD AND COMPUTER-READABLE MEDIUM FOR DRAINAGE MESH OPTIMIZATION IN OIL AND/OR GAS PRODUCING FIELDS

(71) Applicant: PETRÓLEO BRASILEIRO S.A. - PETROBRAS, Rio de Janeiro (BR)

(72) Inventor: MARCO ANTONIO CARDOSO, Rio de Janeiro (BR)

(21) Appl. No.: 18/359,667

(22) Filed: Jul. 26, 2023

(30) Foreign Application Priority Data
Jul. 27, 2022 (BR) 10 2022 014878 3

(51) Int. Cl. G06N 3/126

(52) U.S. Cl. CPC G06N 3/126

(57) ABSTRACT

The present invention proposes the use of an optimization tool based on Genetic Algorithm for the optimization of the drainage mesh, that is, the simultaneous optimization of the quantity, location and length of producing and injecting wells. Said optimization tool provides a robust implementation of a computational method to deal with realistic well positioning problems with arbitrary trajectories, complex models and linear and nonlinear constraints. Said optimization tool uses a commercial reservoir simulator as an evaluation function without using proxies to replace the complete numerical model. A net present value (NPV) calculation is also provided as a criterion for obtaining the optimized drainage mesh.

### FIELD OF THE INVENTION

The present invention pertains to the field of oil and gas engineering. More specifically, the present invention relates to a method of obtaining an optimal drainage mesh for an oil and/or gas producing field.

### BACKGROUND OF THE INVENTION

The drainage mesh of an oil/gas producing field includes defining the number of producing and injecting wells, the location and length of the section of the well open to the flow in the reservoir. The drainage mesh is a key aspect with great impact on a field development project. In this sense, the use of reservoir simulation allows different drainage mesh options to be evaluated. However, manual trial and error procedures that require a lot of experience and knowledge from the team of geoscientists, formed by the engineers, geologists and geophysicists involved in the project, are still used. Considering this, the development of drainage mesh optimization tools that can automate this process is a highly desirable objective.

The optimization of the drainage mesh is a very challenging problem that has received increasing attention due to the need to maximize the recovered volume and minimize production costs to efficiently meet the world demand for oil and gas, in addition to ensuring the implementation of robust projects that are resilient in scenarios of oil price reduction in the global market.

It is known that the development costs of a field are strongly impacted by the number of drilled wells; therefore, it is necessary to optimize the number of wells while maximizing the production and net present value. The search for the best location for locating the wells is a highly non-linear problem that depends on a large number of decision variables. Other constraints, such as the geomechanical behavior of the reservoir, the geometry of the wells, surface facilities and geological and economic uncertainties, can significantly increase the complexity of the problem. Such a complexity can assume another degree of magnitude due to the geometric characteristics of the wells, since horizontal, inclined and, in certain cases, multilateral wells are increasingly common. The number of combinations of possible solutions for well placement variables can grow exponentially. In this way, the search for the optimized solution for this type of problem through the simulation of a few cases, or through the engineer's intuition, becomes impossible.

The common practice in the industry for defining the drainage grid of a field consists of a manual procedure of trial and error that requires a team of qualified and experienced geophysicists, geologists and engineers. Once a drainage mesh proposal has been defined, it must be evaluated through flow simulation, with the dynamic behavior of the wells evaluated in relation to the field production curve and also individually. This approach, due to the significant time to propose a drainage mesh and evaluate the simulation results, limits the number of scenarios that the team can evaluate in order to meet the field production development schedule. In this way, it is expected that the field development strategy proposal does not allow for the maximization of the net present value.

Most research has focused on the performance of the optimization process, trying to minimize the number of runs of the simulation model. However, the technological advance in the development of hardware and software had been changing the application scenario of reservoir simulators. The use of clusters with a large number of CPUs allows several reservoir flow simulations to be executed at the same time, which makes the optimization process, using the detailed simulation model as an evaluation function, a viable task. However, despite the large number of researches on drainage mesh optimization, there is still a lack of robust tools to be applied by the teams involved in development and production projects.

### STATE OF THE ART

Document US20070016389 A1 of Jan. 18, 2007, titled “Method and system for accelerating and improving the history matching of a reservoir simulation model”, inventors DELFINO, Neil Ernest; RONEN, Joshua; VU, Cung Khac, discloses a method and system for determining and graphically displaying the matching error between the production history of a well and/or reservoir and the data simulated by the representative numerical simulation model of the reservoir. In one aspect, document US20070016389 A1 provides a method and software system for utilizing data and associated history matching error analysis to improve predicted recovery of reservoir fluids, which provides an easily understandable synopsis for the fluid reservoir, and which uses history matching error to produce faster and more reliable pressure and production rate predictions for individual wells and/or the reservoir in its entirety. In particular, document US20070016389 A1 includes an interactive “history matching” process to reproduce the historical production and pressure performance of a reservoir to reliably predict its future performance. The more historical data provided for historical matching, the more reliable the US20070016389 A1 “simulation model” becomes, which serves as the basis for determining historical matching errors and the reliability of future performance predictions. As a result, document US20070016389 A1 significantly simplifies and speeds up prior reservoir simulation history matching processes. According to another aspect, US20070016389 A1 provides a method for predicting the recovery of fluids from a reservoir using a computer comprising:
a) receiving history performance data for an individual well and/or reservoir;
b) building a theoretical production output model for the well and/or reservoir using the received data;
c) calculating a history matching error between the theoretical production output model and the historical production output data for said well and/or reservoir;
d) if the calculated value of said history matching error is different from a predetermined value, modifying a parameter of the received data and redo steps (b) and (c);
e) displaying said calculated history matching error as a graphical representation on at least one of a graph and a map; and
f) predicting a future production output for said at least one wellbore and reservoir when the calculated value of the history matching error is equal to or less than the predetermined value.

Document US20070016389 A1 further includes a software system to carry out the above method.

According to another aspect, document US20070016389 A1 provides a method to visually display the calculated history matching error. The visual display allows the user to readily see portions of the well and/or reservoir where there is a substantial discrepancy between historical performance data and simulated performance model data.

According to yet another aspect, document US20070016389 A1 provides a method to provide an improved reservoir simulation model (combined with history), comprising:
a) receiving data for an individual well and/or a reservoir;
b) building a theoretical production output model for said well and/or reservoir using said received data;
c) calculating a history matching error between said theoretical production output model and said historical production output data for said well and/or reservoir;
d) receiving a plurality of reservoir parameters corresponding to a property of said well and/or reservoir.
e) using a neural network to provide a correlation between a selected set of said reservoir parameters and said history matching error;
f) iteratively varying at least one of a selection and a value of said reservoir parameters in the neural network to provide at least one set of reservoir parameters with value(s) that provide a minimum for said history matching error.

Document US20070016389 A1 further includes a software system to carry out the above method.

Document US20020120429 A1 of Aug. 29, 2002, titled “Methods for modeling multi-dimensional domains using information theory to resolve gaps in data and in theories”, discloses methods for modeling multidimensional domains, merging multiple input datasets into a model, applying various dynamic theories to evolve the model, and using information theory to resolve gaps and discrepancies between datasets and theories. More specifically, document US20020120429 A1 models multidimensional domains based on multiple, possibly incomplete and mutually incompatible input datasets, and then uses multiple, possibly incomplete and mutually incompatible theories to evolve the models across time and space. Information theory resolves gaps and conflicts within and between datasets and theories, thereby constraining the range of possible data processes and values. Furthermore, as the information theory approach is based on probability theory, the approach allows for the evaluation of uncertainty in predictions.

One embodiment described in document US20020120429 A1 is a 3D geological basin simulator that integrates seismic inversion techniques with other data to predict the location and characteristics of the fracture. The 3D finite element reaction, transport and basin mechanic simulator includes a rock rheology that integrates continuous poroelastic/viscoplastic, pressure solutions deformation with brittle deformation (fracturing, failure). Mechanical processes are used to co-evolve deformation with multiphase flow, oil generation, mineral reactions and heat transfer to predict the location and producibility of the right fracture points. Information theory uses geological basin simulator predictions to integrate well log, surface, and core data with incomplete seismic data. The geological simulator delineates the effects of regional tectonics, oil-derived overpressure and salt tectonics and builds maps of zones with a high degree of fracture producibility. The method for producing a model of a region of interest in this document comprises:
a) collecting a first set of data points belonging to the region of interest;
b) dividing the first data set into a second data set and a third data set;
c) populating a model with data points from the second data set;
d) interpolating a data point into the model using a subset of data points from the second data set;
e) comparing a subset of data points in the model to a subset of data points in the third data set; and
f) if comparing yields a discrepancy greater than an error threshold, then varying a data point in the model corresponding to a data point in the second data set and repeating the interpolation and comparison.

In a second embodiment, document US20020120429 A1 models a living cell. The cell simulator uses a sequence of DNA nucleotides as input. Using transcription and translation polymerization chemical kinetic rate laws, the cell simulator calculates populations of mRNA and proteins as they evolve autonomously, in response to changes in the environment or from viruses or injected chemical factors. The rules that relate the sequence and function of amino acids and the chemical kinetics of post-translational protein modification allow the cell simulator to capture the autonomous behavior of a cell. A complete set of biochemical processes (including glycolysis, the citric acid cycle, amino acid and nucleotide synthesis) is accounted for with the laws of chemical kinetics. Features such as the prokaryotic nucleoid and the eukaryotic nucleus are treated with a new mesoscopic theory of reaction transport that captures atomic-scale details and corrects for thermodynamics due to the large concentration gradients involved. Metabolic reactions and DNA/RNA/protein synthesis take place in appropriate compartments, while the cell simulator is responsible for active and passive molecular exchange between the compartments.

Document U.S. Pat. No. 8,670,966 B2 of Feb. 18, 2010, titled “Methods and systems for performing oilfield production operations”, discloses techniques for performing oilfield production operations involving an analysis of oilfield production conditions, such as gas-lift configuration, production rates, equipment and other items, and their impact on such operations. Embodiments of methods and systems according to document U.S. Pat. No. 8,670,966 B2 provide techniques capable of optimizing well network production based on a complex analysis of a wide variety of parameters that affect oilfield operations. For example, in one embodiment, a resource allocation method applied across a well network includes receiving topological data into an analytical model of a well network with one or more wells. The topological data include a plurality of performance curves that relate wellbore performance to one or more levels of an applied resource. The method also includes determining an optimal allocation of the applied resource using the analytical model to maximize an operating parameter of the well network, including converting a portion of the analytical model with one or more wells and a linear inequality relation to a modified portion with a single variable and a linear equality constraint. In other aspects, a method may also include coupling an offline solution result with an online solution result during optimal allocation determination. More specifically, it discloses a method to identify an optimal allocation of a resource applied in a well network, comprising:
a) validating, in accordance with a predefined schedule, well test data obtained from one or more wells in the well network by data conditioning and quality checking to generate validated well test data;
b) updating, using a processor and in accordance with predefined programming, a well network model based on validated well test data to generate an updated network model, wherein the well network comprises one or more wells and a surface network, wherein the network model comprises static network parameters, the static network parameters comprising a boundary constraint on well network sources and sinks and the fluid composition, and wherein the boundary constraint and the fluid composition are updated based on validated well test data;
c) updating, in response to a sensitivity analysis of a well test indicating a change in well performance, a well model comprised in the network model based on the validated well test data to generate an updated well model in the updated network model, wherein the sensitivity analysis is performed using the well model to predict a well test parameter value based on other well test parameters, and wherein the well model comprises an lifting configuration of one or more wells and a plurality of performance curves relating the performance well to one or more levels of the applied resource;
d) diagnosing, according to the predefined schedule, the lifting configuration of one or more wells based on the validated well test data and the updated network model to generate a diagnosed lifting configuration; and
e) determining, using the processor and according to the predefined schedule, the allocation of the applied resources using the updated network model based on the diagnosed lifting configuration to maximize an operating parameter of the well network, including:
e1) converting a portion of the updated network model having a linear inequality relation into a modified portion having a single variable and a linear equality constraint; and
e2) solving the modified portion using a modified Newton method, wherein solving the modified portion using the modified Newton method comprises determining one or more inverse derivative curves in order to solve a plurality of Karush-Kuhn-Tucker (KKT) conditions for optimization directly.

Therefore, the technique lacks methods and systems applied to modeling, simulation and optimization determination of the drainage mesh of an oil and/or gas producing field.

### SUMMARY OF THE INVENTION

The present invention proposes the use of an optimization tool based on Genetic Algorithm for the optimization of the drainage mesh, that is, the simultaneous optimization of the quantity, location, and length of producing and injecting wells. Said optimization tool provides a robust implementation of a computational method to deal with realistic well positioning problems with arbitrary trajectories, complex models and linear and nonlinear constraints. Said optimization tool uses a commercial reservoir simulator as an evaluation function without using proxies to replace the complete numerical model.

### BRIEF DESCRIPTION OF THE DRAWINGS

The present invention will be described below with reference to the typical embodiments thereof and also with reference to the attached drawings, in which:

FIG. 1 is a representation of the reference and search populations in the genetic algorithm according to the present invention;

FIG. 2 is a representation of a chromosome of the genetic algorithm according to the present invention;

FIG. 3a is a representation of a well as a chromosome in the genetic algorithm according to the present invention.

FIGS. 3b and 3c are two optimization processes following different criteria for chromosome construction in the genetic algorithm according to the present invention.

FIG. 4a is a representation of a recombination of chromosomes in the genetic algorithm according to the present invention.

FIG. 4b is a representation of a mutation of chromosomes in the genetic algorithm according to the present invention.

FIG. 5 is a representation of an exemplary producing field according to the present invention.

FIG. 6 is a representation of a limited variation in one of the genes on the chromosomes in the genetic algorithm according to the present invention.

### DETAILED DESCRIPTION OF THE INVENTION

Specific embodiments of the present disclosure are described below. In an effort to provide a concise description of these embodiments, all features of an actual implementation may not be described in the specification. It should be appreciated that, in the development of any actual implementation, as in any engineering or design project, numerous implementation-specific decisions must be made to achieve the developers' specific objectives, such as compliance with system- and business-related constraints, which may vary from one implementation to another. Furthermore, it should be appreciated that such a development effort might be complex and time-consuming, but would nevertheless be a routine design and manufacturing undertaking for those of ordinary ability to benefit from the same.

Initially, the concept of genetic algorithm will be reviewed briefly to better situate the reader.

### Genetic Algorithm

Genetic Algorithm is an optimization technique based on analogies with the natural selection of species and genetics, combining concepts such as survival of the fittest individual and random crossing information. Genetic Algorithm modeling consists of encoding each possible solution to a problem in a structure called chromosome, composed of a chain of variables called genes. Each chromosome represents an individual and, in the present context of drainage mesh optimization, each individual represents a viable set of wells for the field under study.

The process of searching for an optimal solution consists of submitting a set of individuals (population) to an evolutionary process that occurs in cycles. Each evolutionary cycle is called a generation and includes the following steps: evaluation, selection, crossover, and mutation.

During the evaluation step, each individual is associated with a fitness value, which quantifies how well the solution represented by that individual solves the problem when compared to other individuals in the population.

In the selection step, a group of individuals is chosen for reproduction based on the fitness values obtained in the evaluation step. The fittest individuals in a generation are more likely to be selected.

The reproduction can be done by one of two different operators: crossover and mutation.

The crossover operation in the crossover step combines genes from two individuals to generate new individuals as a mixture of genes from the original individuals. The mutation operation in the mutation step is applied to separately selected individuals. The mutation operator sets a random value for a gene, so that this operation inserts new genetic information into the population. Thus, simulating the evolution processes of nature, it is possible to assume that, after several generations of evolution, the population will have individuals with better fitness than those of the first generation.

The process of evolution begins with the creation of random individuals to form a population. The new individuals are inserted into the population, removing the least fit from the previous generations. The new population is then evaluated and the cycle (evaluation, evolution) is restarted. A stopping condition of the evolutionary process is the defined maximum number of generations.

### Genetic Algorithms for Restricted Problems

Most of the proposed methods to deal with nonlinear constraints in Genetic Algorithms for optimization problems are based on penalty functions. However, the performance of these methods is highly problem dependent. Also, many methods require additional adjustment of various parameters. In 1995, Michalewicz and Nazhiyath proposed an alternative method called Genocop III (GEnetic algorithm for Numerical Optimization of COnstrained Problems) for problems with linear and non-linear constraints.

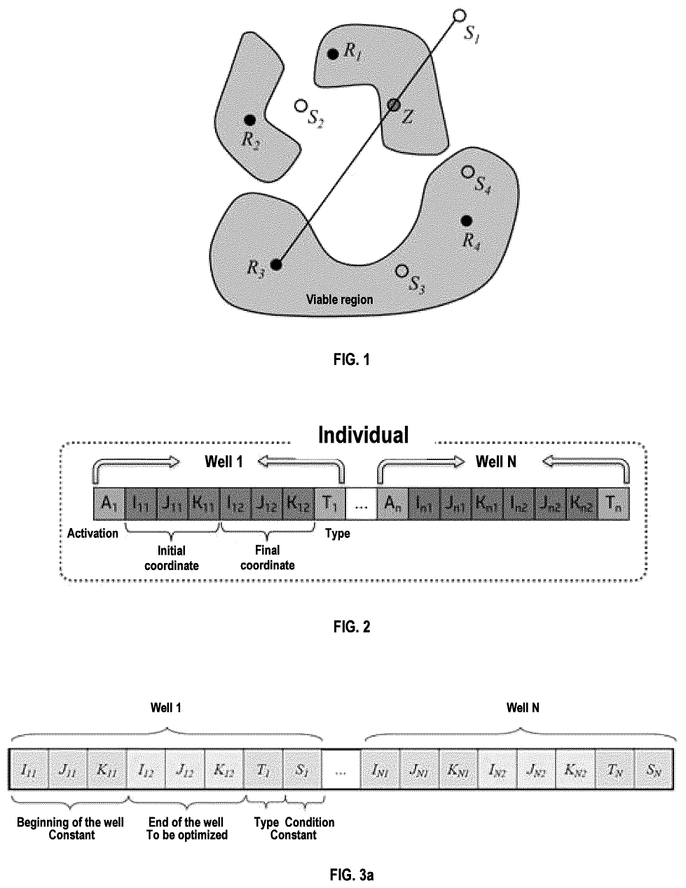

The Genocop III method uses two separate populations, and the evolution in one of them influences the evaluations of individuals in the other. The first population, called the search population, consists of individuals that satisfy the constraints of the problem. The second population, called the reference population, consists only of fully viable individuals, that is, individuals who satisfy all constraints (linear and non-linear). Reference individuals are evaluated directly at the beginning of the optimization process, as are viable search individuals. However, unviable search individuals are “repaired” prior to evaluation. This repair process consists of randomly selecting a reference individual, Ri, and applying a crossover operator between the unviable search individual, Si, and Ri until a new viable individual, Z, is found and then evaluated. If the Z evaluation is better than the Ri evaluation, this individual replaces Ri in the reference population. Depending on the probability, pr, Z can also replace Si. FIG. 1 illustrates the Genocop III procedure.

An exemplary algorithm for obtaining the individual Z according to the Genocop III procedure is provided below:

```
begin
  P = pr; // replacement probability
  if feasible(S) == false
    Z = aS + (1 − a)R; // a ∈ [0, 1]
    while feasible (Z) == false
      Z = aZ + (1 − a)R; // a ∈ [0, 1]
    end while
    if evaluation(Z) > evaluation(R)
      R = Z; // R is replaced by Z
    end if
    if rand( ) ≤ P
      S = Z; // S is replaced by Z
    else
      evaluation(S) = evaluation(Z);
    end if
  end if
end
```

Generalizing the procedures of Genocop III, it is possible to apply the same in different situations. Taking the context of the present invention, it is possible to deal with any number of constraints. For example, and without limiting the invention, the following well positioning constraints in the optimization process can be worked out:
Dimension of the simulation mesh: I_{i,j}∈[1, I_{grid}]; J_{i,j}∈[1, J_{grid}]; and K_{i,j}∈[1, K_{grid}], where I_{grid}, J_{grid} and K_{grid} indicate the number of simulation mesh cells in the i, j and k directions, respectively;
Maximum length of wells: \sqrt{(xᵢ₂−xᵢ₁)²+(yᵢ₂−yᵢ₁)²+(zᵢ₂−zᵢ₁)²}, ≤Lpₘₐₓ, where x and y indicate the Cartesian coordinates referring to the location of the wells;
Minimum distance between wells: Dist(wellᵢ;well_{1 . . . Nwells})≥Dpₘᵢₙss;
Mesh inactive cells: in this case, it is defined that a well cannot start and end in an inactive position of the simulation mesh, but a well can cross inactive cells.

With this set of constraints, the optimization algorithm becomes applicable to reservoir models with non-uniform meshes. These constraints also allow some flexibility in defining the optimization problem. For example, geoscientists can define only a small region of the model and optimize the location of some wells to be drilled, maintaining the minimum distance allowed for already-existing wells in the field.

### Methodology

### Operators

The initial and final rates of an operator of the genetic algorithm are the probabilities of individuals in the population suffering mutation in the gene related to that operator throughout the optimization. When the optimization process starts, the mutation probability will take the value of the initial rate and will approach the final value of the rate linearly, reaching it at the end of the process. The following operators can be used:
Crossover operator: Allows the exchange of genes between individuals;
Mutation operator: Allows mutation in any gene of the individual;
Controlled mutation operator: Allows mutations only in genes related to well location;
Well type operator: Limits the mutation only to the gene related to the well type;
Activation operator: Limits mutation to only the activation gene of the wells.

### Problem Parameterization

The developed methodology defines 8 decision variables to characterize each well. Evidently, the number of variables can be smaller or larger than this without departing from the scope of the invention. In specific applications, the number of variables can be just 1 or it can be a number much greater than 8, with no upper limit, being restricted only by what the person skilled in the art considers relevant in a specific application. For the purposes of this disclosure, without limiting the application, the eight selected variables are divided into:
Three variables that define the initial point of the well in a 3-dimensional map (coordinates I, J and K).
Three variables that define the final point of the well in the 3D map (coordinates I, J and K).
A binary variable that defines the type of well: producing or injecting well.
A binary variable that defines the state of the well: active or inactive.

Due to this parameterization with 8 decision variables per well, the optimization problem becomes large. For example, considering a case with a maximum number of 15 wells, the problem will require 120 variables to be optimized. On the other hand, this parameterization allows the simultaneous optimization of well location, length and number of wells. FIG. 2 shows a schematic representation of the chromosome used in the Genetic Algorithm. A chromosome represents an individual in the population and each individual corresponds to a different well distribution scenario.

In other words, still referring to FIG. 2, the individual N is a chromosome of the genetic algorithm that contains a certain number of genes. For the purposes of the present invention, the N individual/chromosome is the N well in the proposed drainage mesh, which comprises 8 genes, that is, 8 variables that define the initial position, final position, well type and well state. Specifically, the genes/variables I_{N1}, J_{N1} and K_{N1} define the initial position of the well on the map in 3 dimensions, the genes/variables I_{N2}, J_{N2} and K_{N2} define the final position of the well on the map in 3 dimensions, the variable/gene T_{N} defines whether the well is a producing or injecting well, and the variable/gene S_{N} defines whether the well is active or inactive.

In the present description and in most practical applications, the T and S genes of each well can only assume one of two values, for example, 0 or 1, and, after being defined, remain constant throughout all generations. In specific applications, however, these genes can be varied without departing from the scope of the invention.

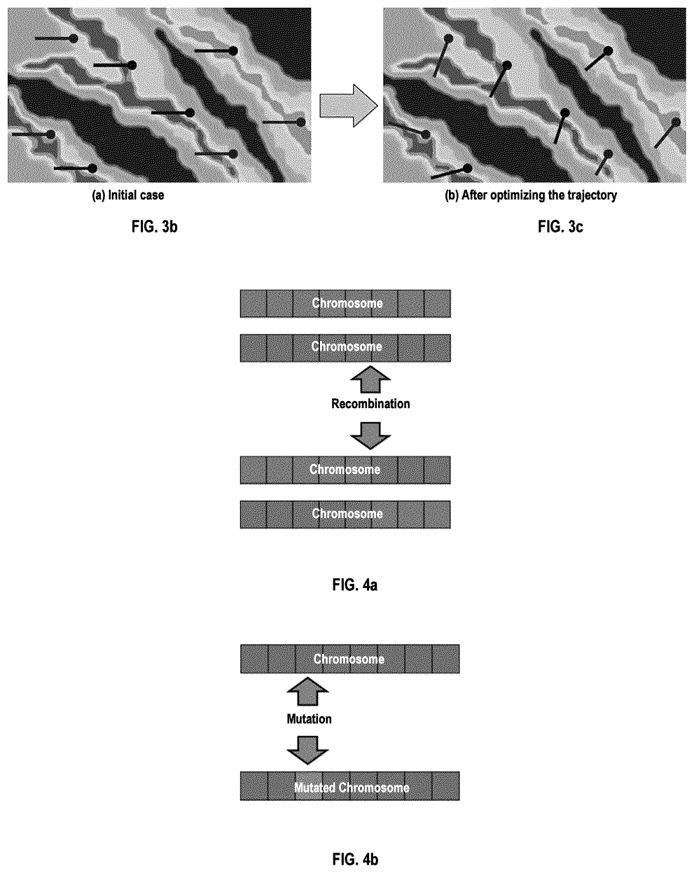

The parameterization also allows flexibility in defining the optimization problem. For example, instead of optimizing all attributes (location, length, type and number of wells), it is possible to optimize only some of the same. One can only optimize the length of the wells, optimizing only the final position of the well and keeping all other decision variables constant. In this case, the problem will actually be smaller, with only 3 decision variables per well. As shown in FIG. 3, only the I₁₂, J₁₂ and K₁₂ coordinates of the final position of the wells are optimized, keeping constant the initial position (I₁₁, J₁₁, K₁₁ coordinates), the well type (producing or injecting well) and the condition (active or inactive). FIG. 3a illustrates an initial case (a), indicating the position of the producing (black) and injecting (blue) wells, with the circles indicating the beginning of each well. According to the example described, the initial position of the wells will be maintained and the final position will be optimized. It is observed that in case (a) of FIG. 3a, initially, all the wells were drilled in the horizontal direction. FIG. 3b illustrates case (b), after the optimization process, where the trajectory of each well is optimized in order to maximize the net present value (NPV) of the field.

In FIGS. 4a and 4b, the effects of two of the operators are illustrated. In FIG. 4a, a representation of the recombination genetic operator is illustrated, where two individuals (chromosomes) are selected to generate one or more individuals by combining the genetic material of the parent individuals. In FIG. 4b, a representation of the genetic mutation operator is illustrated, which allows mutation of the individual (chromosome) through punctual alterations (with low probability of occurrence) usually in just one gene.

As will be seen later, the probability of recombination is high and the probability of mutation is low in the early generations. As the number of generations advances, these probabilities can be changed such that the recombination probability becomes less than the initial recombination probability and the mutation probability becomes greater than the initial mutation probability.

With this approach, several different optimization strategies can be defined, such as: optimizing only producing wells or only injecting wells, optimizing only a group of wells and keeping the other wells constant, optimizing only the number of wells, etc., without being limited to these.

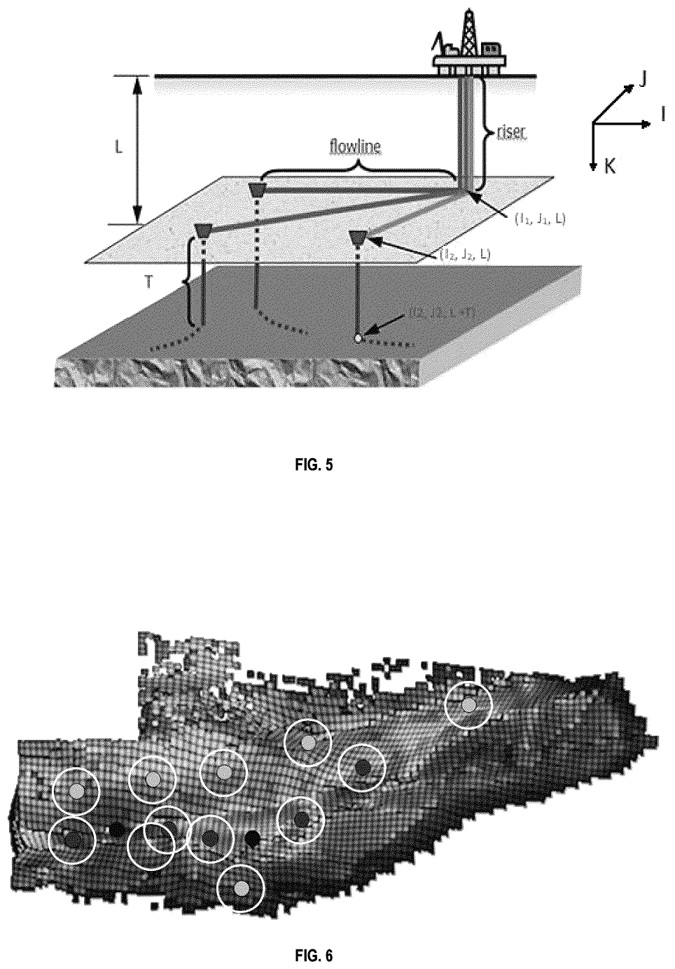

Referring now to FIG. 5, a simplified example mesh is provided for reference. L is the length of the water depth (in the K direction), which must be provided by the user, T is the length of the non-perforated section, which is considered in the fixed cost of the well. The coordinates (I1, J1, L) represent the meeting point of the flowlines coming from the wells with the riser that takes the product to the platform. The initial point of the well open for production is located at coordinates (I2, Y2, L+T) and the initial point of drilling the well is located at coordinates (I2, Y2, L). Of course, although the wells in this example are all vertical, for simplicity, the present invention is not so limited.

### Definition of the Initial Population

The definition of the initial population can impact the performance of the optimization process. It is likely that a poor population of individuals will require several generations to produce a good solution. In this sense, configuring the initial population using the knowledge of the geoscientists responsible for the development of the field is highly desirable, although not mandatory. Advantageously, an initial population configured in a convenient way can demand a smaller number of generations, which optimizes the consumption of time and computational power.

A step in the field development plan is the definition of the types of wells that will be drilled: vertical, directional or horizontal. In the case of horizontal wells, it is necessary to specify the maximum allowable length due to technical limitations. The selection of the best type of well is complex and depends on a series of parameters related to the geological characteristics of the reservoir and the properties of the fluids that will be produced. The maximum number of wells to be drilled depends on the parameters indicated above and the estimated volume of oil and gas for the reservoir under study, as well as the volumes of water and gas that will be injected as a supplementary recovery method, aiming at maximizing the oil and/or gas production.

According to the present method, the genetic algorithm identifies the best configuration of wells (vertical, directional or horizontal) and the number of producing and injecting wells that maximize the net present value of the project.

Depending on the development stage of the field, exploratory wells may have been drilled. When defining the drainage mesh, existing wells can be used to compose the set of wells that will form the field drainage mesh. However, existing wells cannot be part of the optimization process, as their positions are already defined. In this situation, the methodology classifies these wells as “fixed wells”, that is, they are wells that contribute to production, but are not altered by the system.

According to the present method, two different approaches are adopted to generate the initial population: in the first, the user does not provide initial well location scenarios, while in the second approach the user does.

Initial Population without a Drainage Mesh Proposed by the Team

In this first approach, the entire initial population is randomly defined, including the search population and the reference population (which satisfies all linear and non-linear constraints). For this, the genetic algorithm is based on some properties of the simulation model, such as model dimensions, geological properties (porosity and permeability distribution) and estimated oil saturation. With this set of data, the system calculates a map of the distribution of oil thickness in the model, by multiplying the thickness of the cells by the porosity and oil saturation, adding all the cells to one for each Cartesian coordinate i and j in the map of the studied area, thus obtaining the geographical distribution of the most suitable regions for drilling oil and/or gas producing wells. For the injecting wells, the regions with the highest estimates of high injectivity of the injecting wells are mapped, based on the permeability and thickness of the cells. Tests carried out showed that, in the case of using a random population, it is necessary to increase the population size and the number of generations to obtain good results.

Initial Population with One or More Drainage Meshes Proposed by the Team

In this case, the following heuristic is used to generate the search population and the reference population:
Scenarios suggested by the team will represent 10% of the search initial population and the other individuals will be randomly generated; and
The scenarios suggested by the team will represent 50% of the reference population and the other individuals will be randomly generated.

The main objective of this approach is to obtain an improvement of the initial mesh defined by the team. Due to the nature of the method, there is a high probability that, after the optimization process, the final solution will be a descendant of the proposed initial scenario. The better the initial mesh, the greater the probability that the final solution obtained by the optimization process will be similar to the initial mesh.

However, if the proposed initial mesh is not a good initial solution, it can negatively influence the performance of the optimization process, that is, demand a greater number of generations to obtain a satisfactory result. In other words, it would take a greater amount of time and computational power to arrive at a final solution, which would lead to an increase in project costs. In this case, the optimization would probably obtain better results with the first approach, that is, initializing the entire population randomly.

Therefore, it is important that the team responsible for the project evaluates beforehand which approach will be most advantageous.

By default, the genetic algorithm assumes that the optimization will be free, that is, the location of the wells, both producing and injecting wells, will be determined in order to maximize the economic return of the project. Although the optimized case tends to show similarities with the base case, the position of one or more wells can be significantly altered in relation to the proposed initial mesh.

The genetic algorithm also allows the user to limit the displacement of the wells in relation to the proposed locations in the initial mesh. This is advantageous, for example, in cases where the team working on the production development project has carried out a long and meticulous study using geophysics and geology data to propose the initial drainage mesh appropriate to the available data set, aiming at maximizing the recovery and reducing geological risks. In this type of controlled displacement, the maximum displacement in relation to the original positions is specified. For example, the displacement can be limited to 1000 meters in any direction. For example, displacement can be limited to 1000 meters in only one direction, horizontally or vertically on the map. Of course, these are just non-limiting examples. An example of this possibility of variation is illustrated in FIG. 6, where the points represent the initial locations of the wells and the circles that surround the same delimit the area where the new location of each well can be defined.

In this way, the method assumes that the number of wells will not be changed, making changes in the locations of wells within the specified range.

### Optimization Based on Deterministic Simulation

The optimization process is based on a deterministic accomplishment of the simulation model. Practice and several studies show that uncertainties have an important impact on the optimization result. However, due to the significant size of the optimization problem and the computational cost required to perform the same, it may not be economically viable to perform optimizations for a large number of reservoir models. To minimize this problem, the adopted approach is that, before optimization, the team performs an uncertainty analysis, also called risk analysis, as it is known in the art, with an initial well distribution scenario to define a most likely model (P50) and next perform the optimization of the drainage mesh with this P50 model. After that, it is possible to perform another uncertainty analysis to evaluate the impact of uncertainties on the optimization result. Depending on the results, the team might also define a new P50 model and run another optimization. This process can be repeated as many times as necessary.

However, with the increasing use of computer clusters, the use of simulation models obtained in uncertainty analysis studies becomes a reality. In this type of analysis, the uncertainty range of recoverable and/or potentially recoverable volumes can be represented by a probability distribution. In this probability distribution, the following conditions must be satisfied:
There must be at least a 90% probability (P90) that the quantities actually recovered are equal to or greater than the worst estimate.
There must be at least a 50% probability (P50) that the actual quantities recovered are equal to or greater than the best estimate.
There must be at least a 10% probability (P10) that the quantities actually recovered are equal to or greater than the best estimate.

Based on these probabilities, it is possible to optimize the drainage mesh of a field considering simultaneously several accomplishments of the geological model obtained in a probabilistic way. In practical cases, the limitation of the number of geological models used depends on the computational resources available.

### Multiphase Flow Curves

In the development of the production of offshore fields, a drainage mesh composed of producing and injecting wells and pipelines that connect the wells to the production platform is defined. Pipelines are defined in two segments. The first, identified as a production line or flowline, connects the beginning of the well, located at the bottom of the sea, to a position below the platform. The second segment, identified as the riser, connects the flowline vertically to the platform.

Considering that the production or injection of fluids in the reservoir can be located a few thousand meters below the seabed, that the distance from the well to the platform can be a few kilometers and that the depth of the ocean is around 2000 meters, the fluids need to travel distances ranging from 5 to 20 kilometers, approximately. On the path between the reservoir and the platform, the fluids travel through the production line and the riser and, due to the friction between the fluids and the pipelines, pressure drops occur, with an impact on the pressure at which the fluids arrived on the platform. Depending on the operating conditions and characteristics of the fluids, such as flow rate, viscosity, temperature variation, distances to be covered, etc., pressure drop can prevent production from occurring at the necessary pressures and flow rates.

The mentioned pressure drop is modeled through the construction of tables that list various factors of the production line. For example, but not limited to these, such factors could be fluid flow rates; the fraction of gas and water; the distance between the well and the platform; the characteristics of the pipelines, such as roughness, thermal insulation, length; and the pressure at the bottomhole and on the platform. The aforementioned tables can be presented in the form of graphs known as multiphase flow curves. The data that make up the tables are obtained from correlations available in the literature and widely used in the oil/gas industry. The correlations have a very solid theoretical and experimental base, guaranteeing reliability in the modeling of the flow.

In the process of optimizing the drainage mesh, several scenarios of distribution of the set of producing and injecting wells are simulated. During the optimization process, the positions of the wells vary and, to guarantee the accuracy of the simulations, the pressure drop due to the flow between the wells and the platform must be properly treated. It is, therefore, essential to know the position of the platform to determine the length of the production lines. In a production development project, the position of the platform is estimated according to several parameters, for example, sea depth, sea relief, and/or proximity to other marine installations, without necessarily being limited to these.

For the purposes of the present invention, it is necessary to provide multiphase flow curves considering different distances between the reservoir and the platform. During the optimization process, when the genetic algorithm proposes the location of a well, it also determines the distance of that well from the platform and selects the multiphase flow curve closest to that distance. This process is repeated for all wells in the drainage mesh in all simulations performed during optimization.

Multiphase flow curves are widely known in the art, and it is not necessary to go into detail about the same.

### Well Opening Schedule

For the purposes of this description, the optimization of well positioning assumes that all wells start producing and injecting at the same time. There is no schedule optimization. Additionally, production constraints such as bottomhole pressure and fluid production limits are assumed to be constant during the simulation.

The variation of these quantities during the simulation is possible, but would entail an enormous complexity that would burden the computational power and time available. In practical applications, however, it is desirable for these quantities to be constant.

### Optimization Tool Features

### Representation of the Well

The used representation of the well in the optimization process does not explicitly distinguish between vertical, horizontal or deviated wells. All wells are defined as a straight line in 3D space. To accurately represent the well in the simulation model, a routine identifies the simulation mesh cells traversed by each well and calculates the Cartesian coordinates of the entry point and exit point of the well in each cell. This information allows the calculation of the index of the wells so that they are properly treated by the flow simulator.

### Distributed Simulations

Due to the complexity of the optimization problem, in some cases with more than 100 decision variables, the optimization process may require thousands of simulations of the reservoir model. To handle this large number of simulations, the simulations are preferably distributed and managed on a cluster of computers.

### Calculation of Net Present Value

The objective function of the optimization process is the net present value (NPV) of the field development project. Throughout the optimization process, it is possible to observe the growth of the NPV with the progress of the evolutionary process of the genetic algorithm.

In the context of the present invention, a simplified NPV calculation was implemented that considers the following parameters:
Oil and gas sales prices;
Oil, gas and water production costs;
Cost of gas and water injection;
Cost of the production unit (platform);
Relative position between the wells and the platform to calculate the total cost of risers and production or injection lines;
Estimate the cost of wells; and
Taxes and royalties.

The above parameters must be adapted to the production scenario of the field under study. For a developing field, the value of each parameter is based on data from fields with similar characteristics in terms of reservoir and fluid properties, in addition to geographic location and sea depth. In specific applications, one or more of the parameters defined above may be omitted, or still other parameters not mentioned above may be used in combination with one or more of the parameters mentioned here.

The NPV represents the field revenue minus the costs of the wells and the production platform. The revenue is given by the sum of the individual revenue of each well. In the case of producing wells, revenue is a function of the volumes of oil, water and gas produced and the operating cost of processing each fluid. For injecting wells, revenue will be negative, since there will be no revenue from oil and gas production, but the operating costs of injecting water and/or gas. For both producing and injecting wells, the cost of production lines and risers is calculated. The revenue from each well varies over time, being brought to present value through a defined discount rate. The revenue is also subject to taxes and royalties.

The cost of the wells is determined according to the length of the well drilled and the length of the well opened to the reservoir, in addition to the direction of the well (vertical, horizontal or directional) and the cost of drilling, which, in turn, is a function of the direction of the well. The cost of the platform is determined according to the number and flow rate of producing and injecting wells, the equipment needed to treat the produced fluids, energy demand, etc.

### Production Controls

For each simulation, all wells are linked to the same production group. For this group, several production controls can be specified, such as maximum liquid and oil production rate, maximum water injection rate, produced volume replacement control, etc.

### Parameters of the Adaptive Genetic Operator

In general, genetic algorithms produce better results if the crossover and mutation rates are adaptive, that is, being dynamically adjusted as evolution proceeds. Normally, the crossover rate should be high and the mutation rate low at the beginning of evolution, in order to make better use of the initial genetic material of the population, without moving randomly through the solution space. Thus, as the number of generations increases, the population tends to converge, reducing the variety of individuals, which can be increased through the mutation rate, allowing the introduction of greater genetic variation in the population. In the present invention, the crossover rate decreases and the mutation rate increases linearly with advancing generations, starting from an initial value to a final value.

Thus, according to the present invention, a computer-implemented method is provided that relies on the genetic algorithm provided by the Genocop III tool based on the parameters, criteria and operators defined herein. New generations are obtained until a stopping criterion is reached, which is usually a maximum number of generations.

Optionally, the stopping criterion can instead be a threshold value defined for the NPV. In this case, for each obtained generation of individuals (in this example, the wells), the method calculates the NPV according to methods known in the art using the criteria established here, until a generation is obtained that results in a satisfactory NPV.

As mentioned, the method described here is applicable in fields where there is no well, and is also applicable in cases where there is already one or more drilled wells, whether they are in production or not. In this case, these existing wells, called fixed wells, are kept constant during the simulations.

In an exemplary embodiment, the method described here may have the following steps:
1—Receipt of the field simulation model. To consider geological modeling uncertainties, more than one model can be provided, for example, P10 (optimistic), P50 (base) and P90 (pessimistic) models;
2—Base case of the drainage mesh defined by the field development team. If there is no base case, the methodology generates a proposal based on the properties of the geological model;
3—Receipt of the economic parameters used in the construction of the objective function, such as cost of wells, platform cost, operational cost of oil, water and gas production, operational cost of water and gas injection, tax rates, fees and royalties, etc.;
4—If the field already has drilled wells, they are classified as “fixed wells”, since their locations have already been defined;
5—Definition of the platform position by the project team;
6—Receipt of multiphase flow curves, generated by the project team, for different well-to-platform distances, since during the optimization process the positions of the wells vary and, to ensure the accuracy of the simulations, the pressure drop due to the flow between the wells and the platform must be properly treated;
7—Specify whether the optimization will be controlled (maximum displacement of the predetermined wells) or free (the methodology positions the wells aiming at maximizing the economic return);
8—Specify if the wells will be vertical, directional or horizontal (in the case of directional and horizontal wells, it is necessary to specify the maximum length allowed).
9—Specify the maximum number of producing and injecting wells;
10—Specify the parameters of the genetic algorithm (optional, there are pre-defined parameters) and the maximum number of generations for the evolution of the population; and
11—At the end of the optimization process, the user has access to the proposed drainage mesh that obtained the highest net present value (NPV), as well as the NPV of each well.

In specific embodiments, one or more of the above steps may be omitted. In other words, the method described herein has a high degree of customization.

Advantageously, the method here allows obtaining deeply sophisticated drainage meshes, which would be extremely difficult or even impossible to obtain through conventional analysis methods.

As a corollary, the method described here allows to maximize oil/gas production while minimizing man-hour costs that would be dedicated to obtaining a drainage mesh that would nevertheless be less efficient.

Advantageously, in applications where there are fixed wells, whether exploratory or not, the method described herein contributes to an increase in production, consequently increasing the NPV.

In a further embodiment of the present invention, a non-transient computer-readable medium is provided. The medium can be, for example, a memory, a flash memory, a hard disk, a compact disk, or any other device capable of storing computer instructions. When the readable medium of the present embodiment is read by a computer, the computer is enabled to perform the drainage mesh optimization method in oil and/or gas producing fields as described above.

Although the aspects of the present disclosure may be susceptible to various modifications and alternative forms, specific embodiments have been shown by way of example in the drawings and have been described in detail herein. However, it should be understood that the invention is not intended to be limited to the particular disclosed forms. Rather, the invention must encompass all modifications, equivalents and alternatives that fall within the scope of the invention as defined by the following attached claims.

1. A computer-implemented method for optimizing the drainage mesh in oil and/or gas producing fields, the method comprising the steps of:
a) obtaining a current drainage mesh from an initial drainage mesh;
b) obtaining a new generation of the drainage mesh from the current drainage mesh by means of a genetic algorithm, wherein the new generation of the drainage mesh becomes the current drainage mesh; and
c) repeating the step (b) until a stopping criterion is reached,
wherein, in the genetic algorithm, each chromosome represents a well to be positioned in the producing field.

2. The method of claim 1, wherein the initial drainage mesh is provided by a user.

3. The method of claim 1, wherein the initial drainage mesh is randomly generated by the genetic algorithm.

4. The method of claim 1, wherein the initial drainage mesh has one or more fixed wells.

5. The method of claim 1, wherein step (b) is performed based on one or more multiphase flow curves of the producing field provided by a user.

6. The method of claim 1, wherein at least one gene on at least one of the chromosomes in the genetic algorithm is kept fixed or within a predefined range.

7. The method of claim 1, wherein step (a) additionally comprises providing a maximum number of wells to be generated provided by the user.

8. The method of claim 1, wherein each well is a producing well or an injecting well.

9. The method of claim 8, wherein the maximum number of wells to be generated includes a maximum number of injecting wells and/or a maximum number of producing wells.

10. The method of claim 1, wherein each well can be a horizontal, vertical or directional well.

11. The method of claim 1, wherein step (b) comprises calculating the net present value (NPV) of the new drainage mesh based on predefined parameters.

12. The method of claim 11, characterized in wherein the stopping criterion is either reaching a predefined maximum number of generations or the NPV reaching a predefined value.

13. The method of claim 11, wherein the NPV calculation parameters include one or more of the cost of wells, platform cost, operational cost of oil, water and gas production, operational cost of water and gas injection, tax rates, fees, royalties.

14. The method of claim 1, wherein the stopping criterion is a maximum number of repetitions of the step (b).

15. A computer-readable non-transient storage medium comprising instructions stored therein, characterized in that the instructions, when read by a computer, cause the computer to perform the steps of:
a) obtaining a current drainage mesh from an initial drainage mesh;
b) obtaining a new generation of the drainage mesh from the current drainage mesh by means of a genetic algorithm, wherein the new generation of the drainage mesh becomes the current drainage mesh; and
c) repeating the step (b) until a stopping criterion is reached,
wherein, in the genetic algorithm, each chromosome represents a well to be positioned in the producing field.
